(19) United States
(12) Patent Application Publication
(10) Pub. No.: US 2007/0161930 A1
(43) Pub. Date: Jul. 12, 2007

(54) SIZING INSTRUMENT FOR A BODILY JOINT SUCH AS AN INTERVERTEBRAL DISC SPACE

(76) Inventors: Cliff Reitzig, Riedheim-Weilheim (DE); Stephan Eckhof, Riedheim-Weilheim (DE)

Correspondence Address: DICKE, BILLIG & CZAJA, FIFTH STREET TOWERS, 100 SOUTH FIFTH STREET, SUITE 2200, MINNEAPOLIS, MN, 55402, US

(21) Appl. No.: 11/563,076

(22) Filed: Nov. 24, 2006

Related U.S. Application Data
(60) Provisional application No. 60/739,860, filed on Nov. 23, 2005.

(51) Int. Cl. A61B 5/103

(52) U.S. Cl. 600/594

(57) ABSTRACT

A sizing instrument for measuring a size of a bodily joint, such as an intervertebral disc space. The instrument includes an expansion assembly, a handle assembly, and an indicator assembly. The expansion assembly includes a movable member that is transversely transitionable relative to a reference member between a first position and a second position. The handle assembly actuates the moveable member. The indicator assembly includes an indicator adapted to communicate a dimensional measurement associated with the second position of the moveable member.

### REFERENCE TO RELATED APPLICATION

The subject matter of this application is related to the subject matter of U.S. Provisional Patent Application Ser. No. 60/739,860, filed Nov. 23, 2005 and entitled “Measuring Device for Intervertebral Space and Method,” priority to which is claimed under 35 U.S.C. §119(e) and an entirety of which is incorporated herein by reference.

### BACKGROUND

The present invention relates to surgical measurement or sizing instruments and methods. In particular, the present invention relates to instruments and methods for measuring or sizing bodily joints, such as intervertebral disc spaces.

Various surgical procedures entail the need for estimating a size of an enclosed bodily joint, and typically require the use of one or more instruments. For example, prior to implanting a device into an intervertebral disc space/joint, a sizing instrument is normally employed to first estimate the size of the disc space so that an appropriately sized implant can be selected. Sizing instruments for measuring a size of a spinal disc space typically include a distal end that approximates a size and shape of an implant to be inserted into the disc space. Often times, the sizing instrument is impacted into the disc space with a mallet or other tool. A sequential sizing method (i.e., smallest to largest) is typically used, with the sizes of distal ends of the sizing instrument graduating until a desired fit is achieved. The desired fit is determined by a user based upon tactile feel, for example by determining whether the fit of the sizer in the disc space “feels” not too loose and not too tight. Based upon this subjective “feel test,” a final sizer is selected as an indicator of an appropriate implant size for a particular patient. Similar techniques are employed for estimating or measuring the size of other bodily joints.

Unfortunately, tactile feel is subjective and varies from person-to-person. Such directions as snug, or not too tight, while generally appropriate, leave room for some individual error. While surgeons have become adept at the sizing method described above, ensuring proper sizing techniques is important. For example, excessive impaction might be used during sizing to drive the distal end of the sizer instrument into the intervertebral disc space (or other bodily joint). As a result, the vertebrae could be over-distracted, resulting not only in damage to vertebral body endplates (or other bodily tissue or structure), but also damaging soft tissue stabilizers, which can result in an increase in iatrogenic instability, for example.

Another surgical concern is the potential damage imparted upon structure(s) of the joint during surgery. For example, an intervertebral disc generally consists of a nucleus pulpous (“nucleus”), annulus fibrosis (“annulus”) and two, opposing vertebral end plates. The normal annular plies act to keep the annulus tight about the nucleus. During discal surgery, a surgical knife or tool is used to completely sever some portion of the annulus and/or remove an entire section or a “plug” of the annulus tissue. The size of such a plug might be determined according to the space requirements of a particular measurement tool used to estimate the size of the intervertebral space. When an entire section of the annulus is cut or removed, the layers making up the annulus “flay” and/or “pull back” and the constraining or tightening ability of that portion of the annulus is lost. Further, the chances of the annulus healing with restoration of full strength are greatly diminished, while the likelihood of nucleus herniation is increased. An even greater concern arises where a significant portion of the annulus is removed entirely. A more desirable solution is to leave as much of the annulus intact as possible during and after implantation, and thus reducing size of the annulus opening required for insertion of the sizing instrument. Similar concerns exist for sizing or evaluating other bodily joints.

### SUMMARY

Some aspects in accordance with principles of the present disclosure relate to a sizing instrument for measuring a bodily joint bounded by top and bottom surfaces, such as an intervertebral disc space. The sizing instrument includes an expansion assembly, a handle assembly, and an indicator assembly. The expansion assembly includes a reference member and a movable member. The movable member is transversely transitionable relative to the reference member from a first position to a second position. The handle assembly maintains the expansion assembly and is adapted to actuate the movable member between the first and second positions. The indicator assembly is associated with a proximal portion of the handle assembly and is adapted to communicate a dimensional measurement associated with the second position of the expansion member. In some embodiments, the handle assembly is configured to translate rotational movement of a handle thereof into a longitudinal movement that in turn is applied to the expansion assembly.

Other aspects of the present disclosure relate to a method of measuring an interior of a bodily joint, such as an intervertebral disc space. The method includes providing the instrument described above. The movable member is collapsed relative to the reference member and is disposed in the bodily joint otherwise having a dimension. The movable member is expanded against a boundary of the bodily joint to measure the dimension. The dimension is communicated to a user via the indicator assembly. With this technique, a wide variety of bodily joints can be sized in a minimally invasive manner.

### BRIEF DESCRIPTION OF THE DRAWINGS

FIG. 1 is a perspective view of one embodiment of a sizing instrument according to principles of the present invention;

FIG. 2 is an exploded, perspective view of the instrument of FIG. 1;

FIG. 3 is a perspective view of an expansion member of the instrument of FIG. 1;

FIG. 4 is a perspective view of a piston of the instrument of FIG. 1;

FIG. 5 is a perspective view of a measuring tip of the instrument of FIG. 1;

FIG. 6 is a front view of the measuring tip of FIG. 5;

FIG. 7 is a back view of the measuring tip of FIG. 5;

FIG. 8 is a perspective view of an expansion assembly of the instrument of FIG. 1;

FIG. 9 is a front, cross-sectional view of the expansion assembly of FIG. 8;

FIG. 10 is an exploded, perspective view of a guide assembly of the instrument of FIG. 1;

FIG. 11 is a perspective view of a main handle of the instrument of FIG. 1;

FIG. 12 is a front, cross-sectional view of the main handle of FIG. 11;

FIG. 13 is a perspective view of a drive tube of the instrument of FIG. 1;

FIG. 14 is another perspective view of the drive tube of FIG. 13;

FIG. 15 is a top view of a translation shaft of the instrument of FIG. 1;

FIG. 16 is a perspective view of an adjustment handle of the instrument of FIG. 1;

FIG. 17 is a back, cross-sectional view of the adjustment handle of FIG. 16;

FIG. 18 is a perspective view of an insert of the instrument of FIG. 1;

FIG. 19 is front, cross-sectional view of the insert of FIG. 18;

FIG. 20 is a perspective view of a scale cap of the instrument of FIG. 1;

FIG. 21 is another perspective view of the measuring cap of FIG. 20;

FIG. 22 is a front, cross-sectional view of the scale cap of FIG. 20;

FIG. 23 is a back view of a scale pointer of the instrument of FIG. 1;

FIG. 24 is a perspective view of the scale pointer of FIG. 23;

FIG. 25 is a cross-sectional view of the instrument of FIG. 1;

FIGS. 26-28 are top views of a distal portion of the instrument of FIG. 1 in various states of expansion; and

FIG. 29A is a top view of the distal portion of the instrument of FIG. 1 disposed in an intervertebral disc space; and

FIG. 29B is a simplified view of a proximal portion of the instrument, illustrating a measurement indication of a dimension associated with the disc space of FIG. 29A.

### DETAILED DESCRIPTION

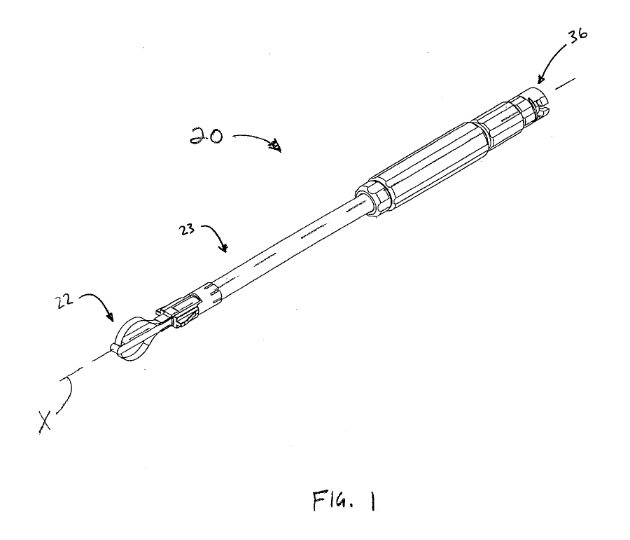

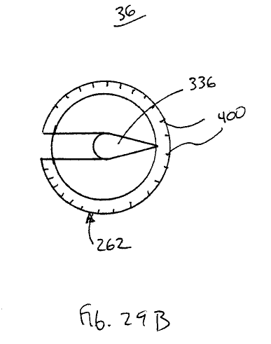

One embodiment of a sizing instrument 20 adapted for measuring bodily joint, such as an intervertebral disc space, is shown for general reference, and in an assembled form, in FIG. 1. Generally speaking, the instrument 20 adapted for measuring internal dimension(s) of a bodily joint 500 (FIG. 29A), for example height and width. Methods of sizing or measuring with the instrument 20, as well as advantages achieved with the instrument 20 and methods of use thereof, should be understood with reference to the text that follows.

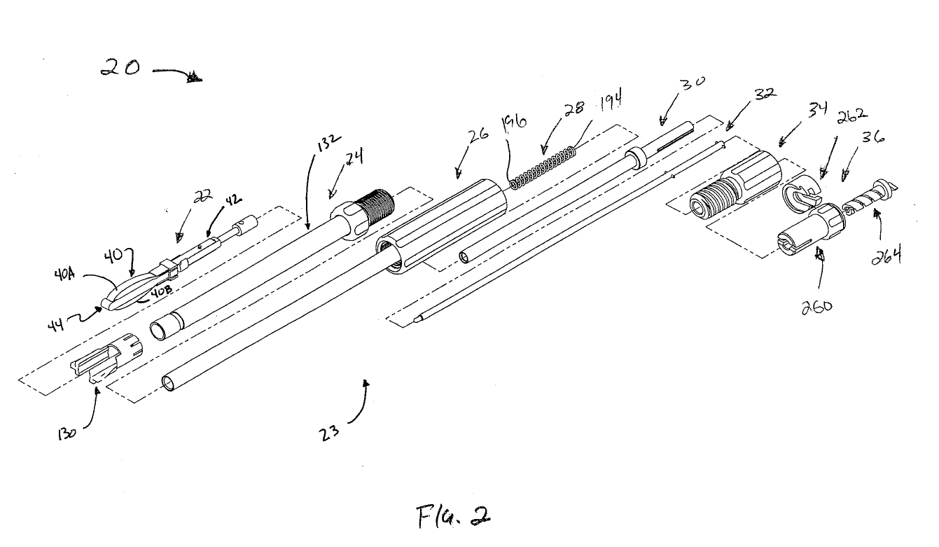

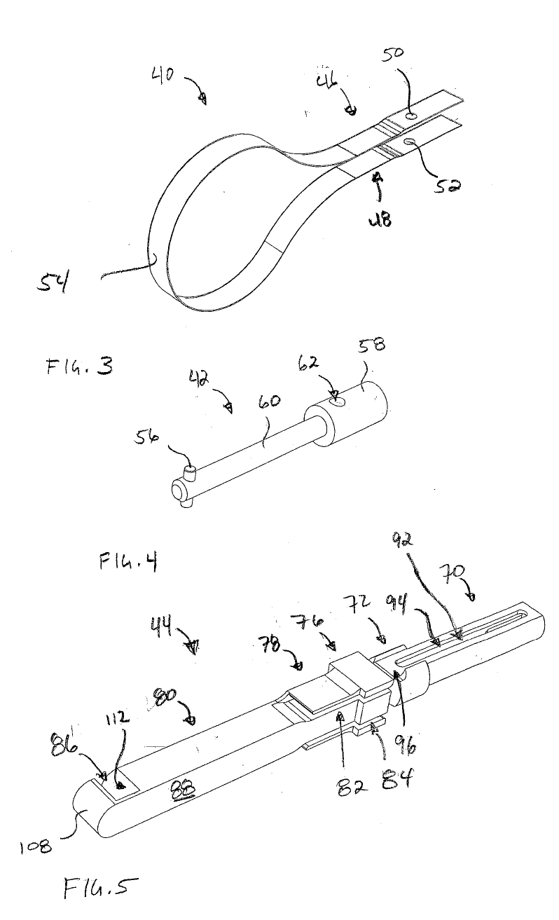

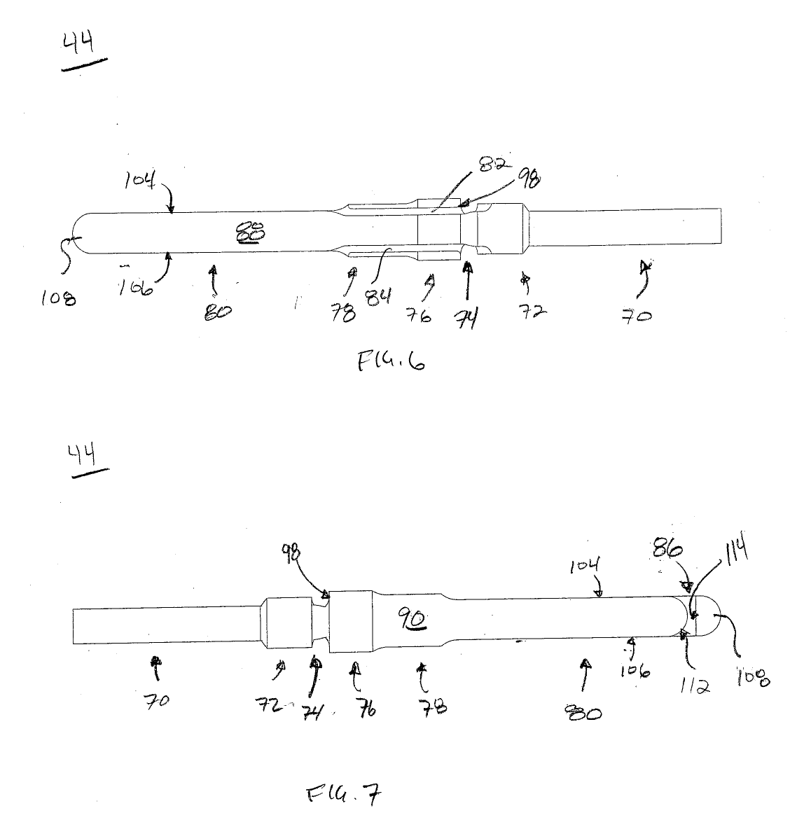

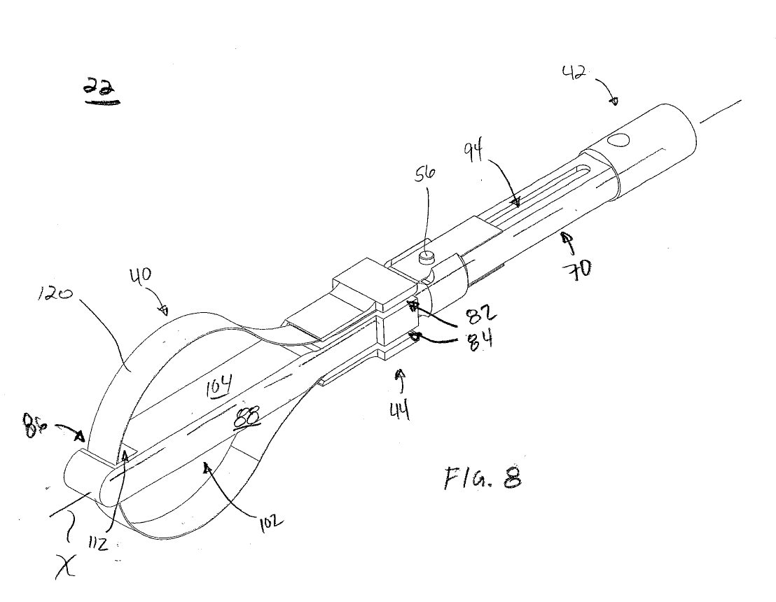

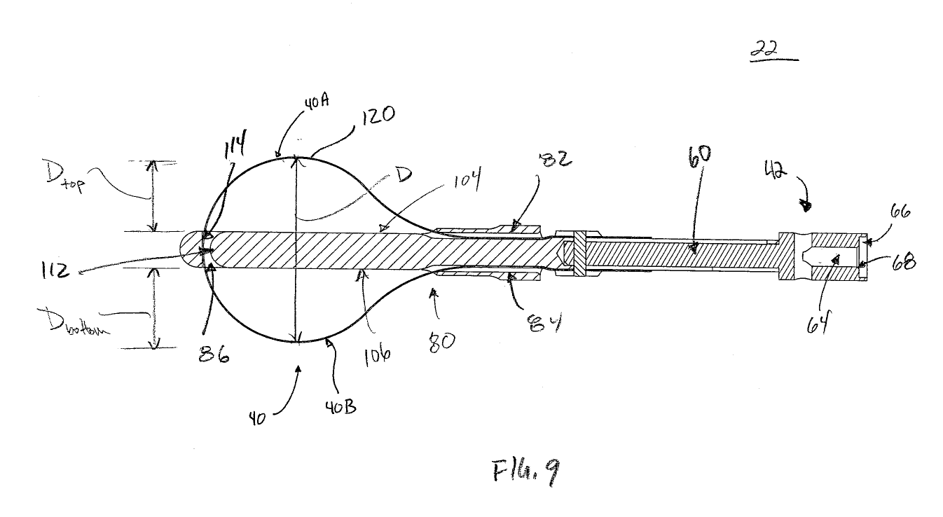

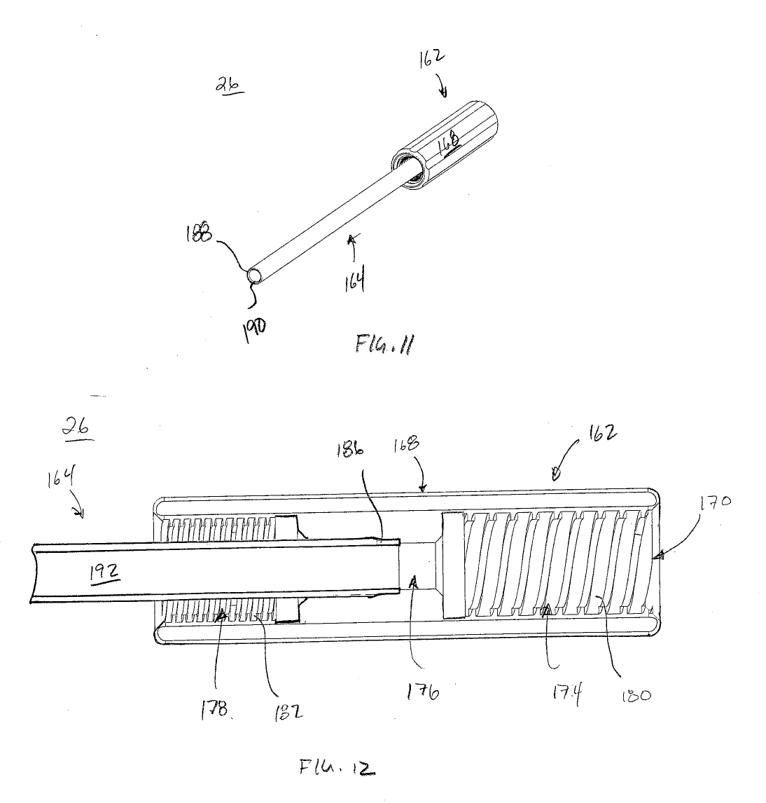

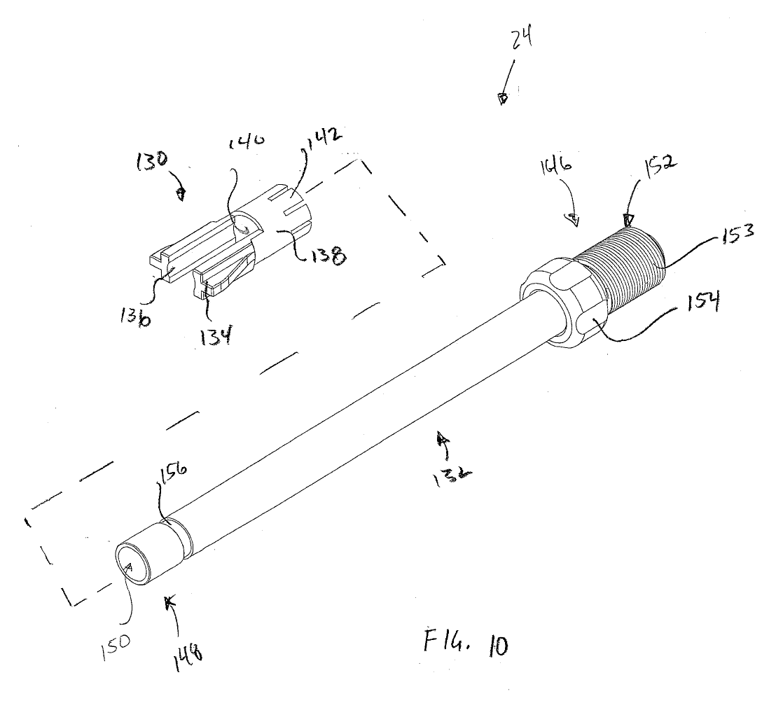

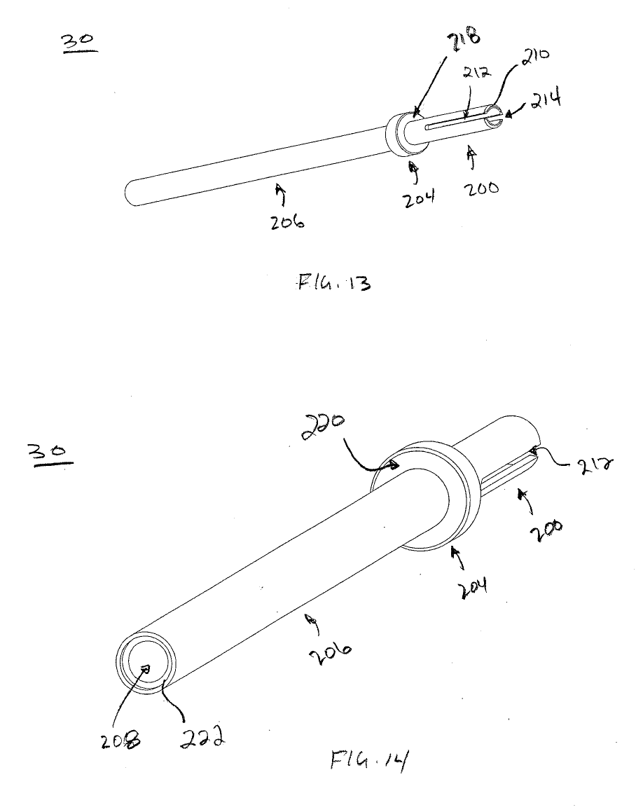

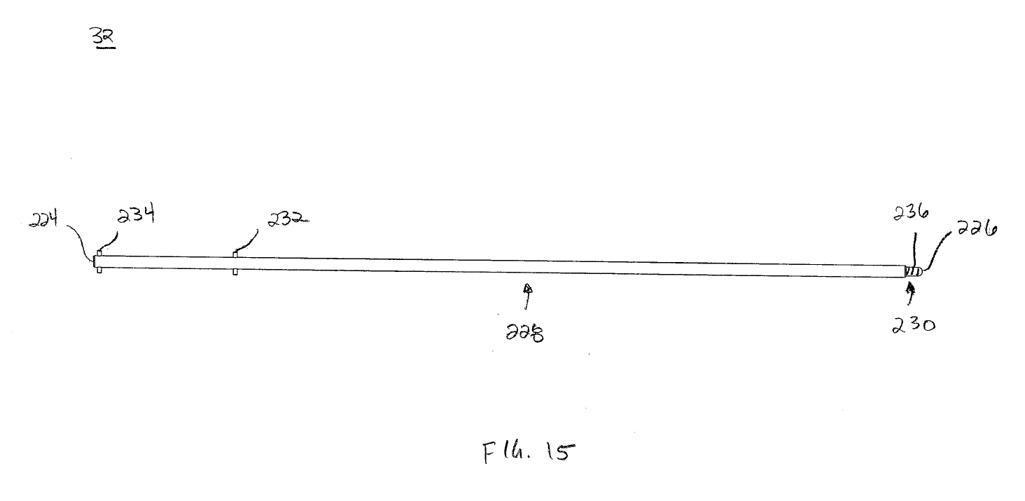

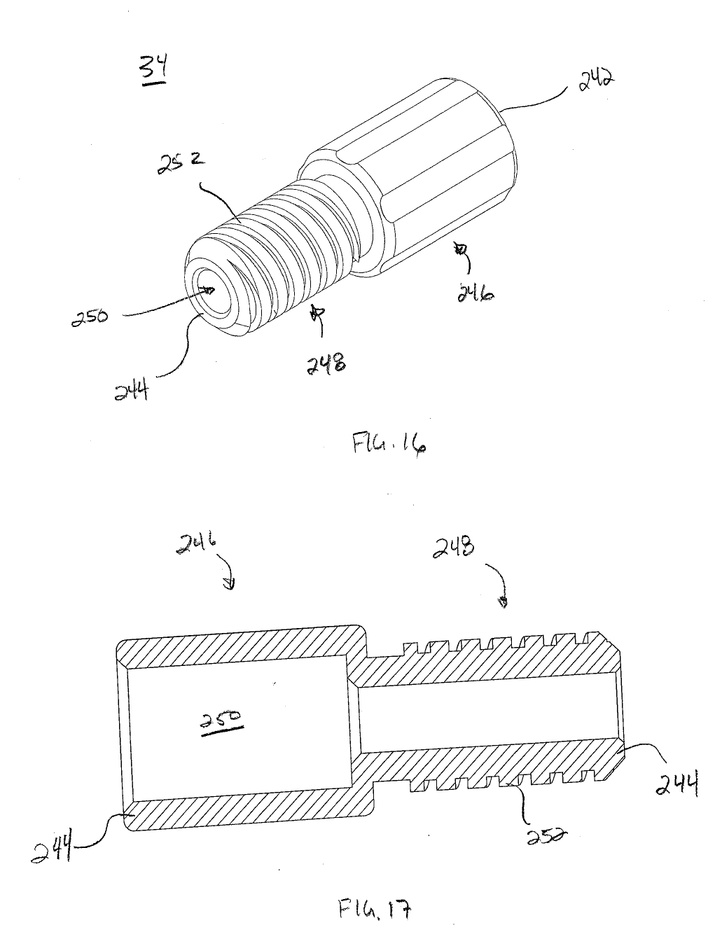

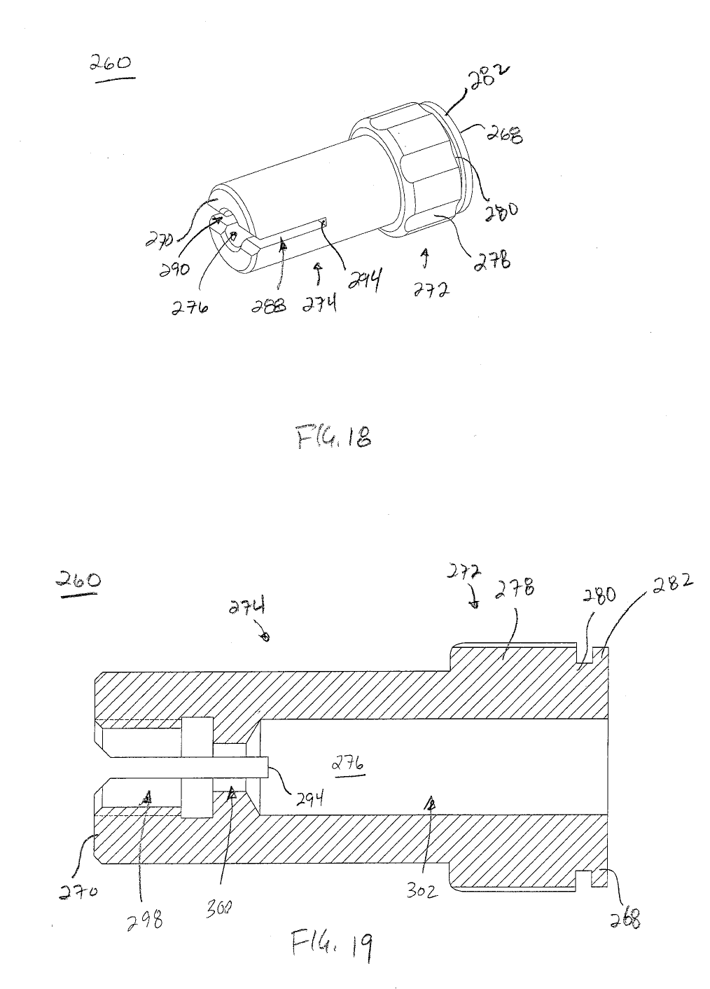

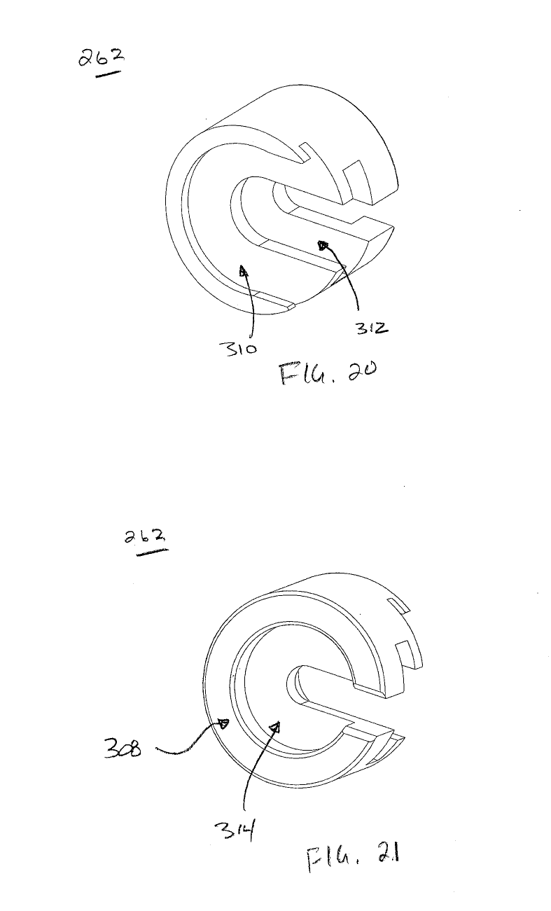

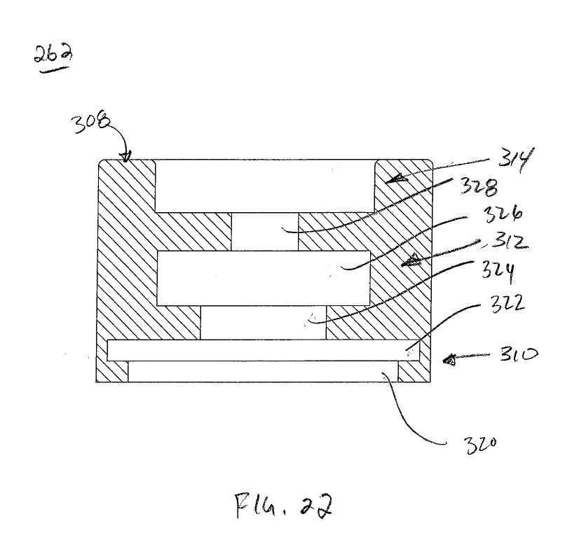

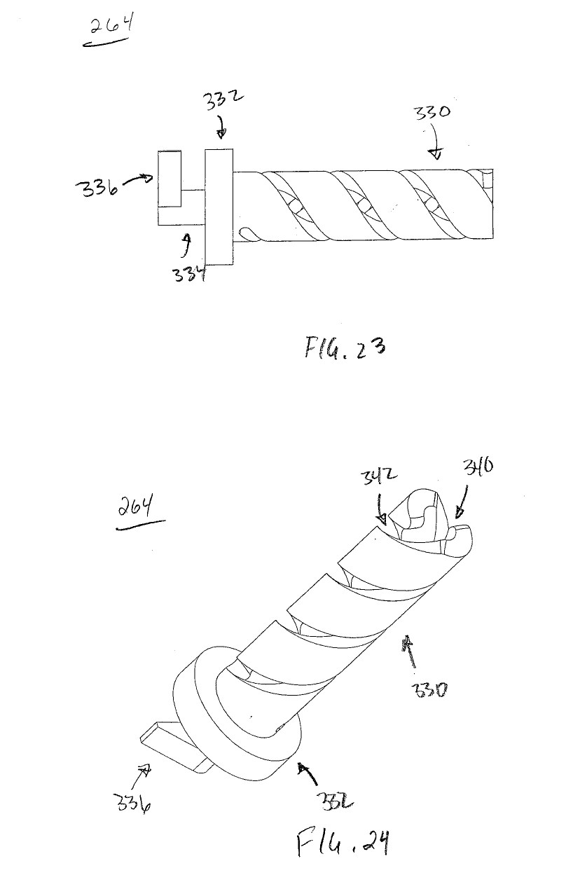

FIG. 2 illustrates the instrument 20 in an exploded configuration to show the various embodiment components of the in greater detail. With reference to FIG. 2, the instrument 20 includes an expansion assembly 22, a handle assembly 23, and an indicator assembly 36, and defines a central longitudinal axis X (FIG. 1), along which each of the above-referenced components is coaxially disposed. In general terms, the handle assembly 23 maintains, and controls actuation of, the expansion assembly 22 in performing a joint measuring operation. The indicator assembly 36 is also associated with the handle assembly 23, and provides a visual indication of a particular dimension reflected by the expansion assembly 22. In one embodiment, the sizing instrument 20 defines an overall length of about 12 inches, although other dimensions are equally acceptable in other embodiments.

The expansion assembly 22 includes a movable member 40, a piston 42, and a reference member 44. In general terms, the movable member 40 is, upon final assembly, transversely movable relative to the reference member 44 (e.g., via movement of the piston 42). A distance of movement or, in some embodiments, expansion of the movable member 40 relative to the reference member 44 is indicative of a dimension of a particular confined space being measured or evaluated.

With reference to FIG. 3, the movable member 40 is in some embodiments an expansion member, defines a length, a width, and a thickness, and extends from a first end portion 46 to a second end portion 48. The expansion member 40 can form or include a first hole 50 in the first end portion 46 and a second hole 52 in the second end portion 48. The expansion member 40 can be formed of a substantially flexible material and/or is relatively thin, such that the expansion member 40 can be flexibly bent and re-bent, or deflected, in a lengthwise direction, without substantial plastic deformation (e.g., a metal “tape”). However, in one embodiment, the expansion member 40 resists bending or flexing in a widthwise direction, i.e. in a direction of the width. With these configurations, the expansion member 40 can be in the form of a strip, wire, cable, etc., having a substantially rectangular shape in lateral cross-section (e.g., within 15% of a true rectangle); alternatively, a number of other lateral cross-sectional shapes are also acceptable (e.g., substantially circular (e.g., within 15% of a true circle), irregular shape, etc.). In one embodiment, the expansion member 40 is about 0.15 mm thick, 5 mm wide, and 100 mm long, although other dimensions are equally acceptable in other embodiments. The expansion member 40 can be formed of metallic alloys, although other materials, for example polymeric materials, and super elastic alloys are also contemplated. While the expansion member 40 is shown as being a continuous body, in other embodiments, the expansion member 40 can consist of two or more, separately provided bodies that are subsequently assembled to the reference member 44 as described below.

With reference to FIG. 4, the piston 42 includes a pin 56, a collar 58 and a rod 60 extending proximally from the collar 58. The collar 58 is substantially cylindrical in shape. As best shown in FIG. 9, the collar 58 forms an inner cavity 64 and defines a spring seat 66 and a translation shaft seat 68 opposite the rod 60. The collar 58 also forms a plurality of female threads (not shown) along a face of the cavity 64. As will be described below, the cavity 64 is female-threaded and includes the female threads such that the cavity 64, as well as the spring seat 66 and the translation shaft seat 68, are adapted to receive corresponding components of the handle assembly 23.

Returning to FIG. 4, the rod 60 is substantially cylindrical in shape and defines an outer diameter. The rod 60 has a hole (hidden in FIG. 4) extending transversely there through, sized to receive the pin 56 as shown. As will be described in greater detail below, the pin 56 assists in securing the expansion member 40 to the piston 42, and the piston 42 within the reference member 44. In some embodiments, the rod 60 defines a length on the order of 40 mm, although other dimensions are equally acceptable in other embodiments.

With reference to FIG. 5, the reference member 44 can, in some embodiments, be described as a measuring tip, is substantially continuously formed, and includes a piston housing 70, a first collar 72, a transition portion 74 (FIG. 6), a second collar 76, a neck 78 and a guide member 80. In some embodiments, the measuring tip 44 is substantially straight. However, in other embodiments, the measuring tip 44 is optionally angled and/or curved to allow for ease of surgical placement and/or visualization. The measuring tip 44 forms a top guide slot 82, a bottom guide slot 84, and an end guide slot 86, which, as will be described below, are formed to receive the expansion member 40. Additionally, the measuring tip 44 defines a first side 88 and a second side 90 opposite the first side 88.

The piston housing 70 has an inner lumen 92 (shown partially obscured in FIG. 5) formed to coaxially receive the rod 60 of the piston 42. The piston housing 70 has a top channel 94 and a bottom channel (not shown). Both the top channel 94 and the bottom channel are open to the inner lumen 90. In particular, the top channel 94 and the bottom channel are formed to slidably receive the pin 56 of the piston 42 as the piston travels proximally or distally through a piston stroke.

The first collar 72 is substantially cylindrical and is formed about the piston housing 70. The first collar 72 defines a substantially circular, transverse cross-section having an outer circumference. The first collar 72 is male-threaded and forms a plurality of male threads (not shown) encircling the outer circumference. While the first collar 72 is substantially cylindrical in one embodiment, the first collar 72 also has a top slot 96 and a bottom slot (not shown) cut into the outer circumference. In particular, both the top slot 96 and the bottom slot are substantially flat and formed longitudinally along the first collar 72. As will be described in greater detail below, the plurality of male threads are configured to mate with a component of the handle assembly 23, while the top slot 96 and bottom slot are formed to slidably receive the first and second portions 46, 48 of the expansion member 40.

FIGS. 6 and 7 show the measuring tip 44 from front and back views, respectively. Referring to FIGS. 6 and 7, the transition portion 74 defines a transition between the first collar 72, and the second collar 76. Along these lines, the transition portion 72 tapers in width and thickness relative to the first collar 72 and the second collar 76.

Returning to FIG. 5, the second collar 76 defines a substantially rectangular transverse cross-section and a proximal face 98 (indicated, but not shown in FIGS. 6 and 7). As will be described in greater detail below, the proximal face 98 is sized and shaped to abut against a portion of the handle assembly 23. The neck 78 extends distally from the second collar 76. The neck 78 also defines a substantially rectangular transverse cross-section and tapers in width and thickness relative to the second collar 76.

The guide member 80 extends distally from the neck 78 and defines a top face 104, a bottom face 106 (indicated generally in FIGS. 6 and 7) opposite the top face 104, and a distal tip 108. Generally, the guide member 80 defines a substantially rectangular transverse cross-section having a width and a thickness. In one embodiment, the top and bottom faces 104, 106 are substantially planar, while the distal tip 108 is substantially rounded.

Returning again to FIG. 6, the top guide slot 82 is formed through the second collar 76 and the neck 78. In one embodiment, the top guide slot 82 extends from the proximal face 98 of the second collar 76, through the second collar 76 and the neck 78, and to the top face 104 of the guide member 80 where the neck 78 transitions to the guide member 80. In turn, the bottom guide slot 84 is also formed through the second collar 76 and the neck 78. In one embodiment, the bottom guide slot 84 extends from the proximal face 98 of the second collar 76, through the second collar 76 and the neck 78, and to the bottom face 106 of the guide member 80 where the neck 78 transitions to the guide member 80. The top and bottom slots 94 are both open to the first side 88 of the measuring tip 44, and are both bounded by the second side 90 (FIG. 7) of the measuring tip 44.

With reference to FIG. 7, the end guide slot 86 is formed through the thickness of the guide member 80 proximate the distal tip 108. The end guide slot 86 defines a proximal end face 112 (FIG. 5) that is substantially rounded, and a distal end face 114 (referenced generally in FIG. 7) that is substantially flat. In one embodiment, the end guide slot 86 is open on the second side 90 of the measuring tip 44 and is bounded by the first side 88 (FIG. 6). As will be described in greater detail below, the end guide slot 86 is configured to receive the expansion member 40.

FIG. 8 illustrates the expansion assembly 22 in an assembled form from a perspective view. The piston 42, and in particular the rod 60 (FIG. 5) of the piston 42, is coaxially inserted into the inner lumen 92 (FIG. 5) of the piston housing 70. In turn, the pin 56 of the piston 42 is assembled to the rod 60 and extends through the top channel 94 and the bottom channel (not shown) of the slide member 76. In operation, the pin 56 allows the piston 42 to move distally and proximally as the pin 56 travels in the top channel 94 and the bottom channel. It should also be understood that in one embodiment, the pin 56 also helps prevent inadvertent rotation and/or ejection of the rod 60 from the inner lumen 80.

The expansion member 40 is connected to the piston 42 and maintained by the measuring tip 44 such that actuation or movement of the piston 42 effectuates transverse transitioning or movement (e.g., expansion and collapse) of the expansion member 40 relative to (e.g., outwardly away from, or inwardly toward) the measuring tip 44. In one embodiment, the pin 56 of the piston 42 is secured in the first and second holes 50, 52 of the expansion member 40. So assembled, the first end portion 46 extends through the top guide slot 82 of the measuring tip 44 and the top channel 94 of the first collar 72. In turn, the second end portion 48 of the expansion member 40 extends through the bottom guide slot 84 of the measuring tip 44 and the bottom channel (not shown) of the first collar 72. With the first and second end portions 46, 48 slidably maintained in the top and bottom guide slots 82, 84 the expansion member 40 loops through the end guide slot 86.

Furthermore, in one embodiment, an opposing nature of the top and bottom guide slots 82, 84 relative to the end guide slot 86 assist in maintaining the expansion member 40 in the guide slots 82, 84, 86. In particular, the end guide slot 86 is open to the second side 90 of the measuring tip 44, while the top and bottom guide slots 82, 84 are open to the first side 88 of the measuring tip 44. With the expansion member 40 bounded by the second side 90 at the top and bottom guide slots 82, 94, and bounded by the first side 88 at the end guide slot 86, inflexibility of the expansion member 40 when bent widthwise helps prevent inadvertent ejection of the guide member 40 from the guide slots 82, 84, 86. However, other manners of retaining the expansion member 40 are contemplated. For example, in another embodiment, a member connects across the end guide slot 86 on the second side 90 of the measuring tip 44. Additionally, the measuring tip 44 optionally includes radio-opaque markers to assist if positioning under fluoroscopy.

FIG. 9 illustrates the expansion assembly 22 from a front, cross-sectional view along the longitudinal axis X (FIG. 1). With reference to FIG. 9, and in view of the above-described relationships, it should be understood that the expansion member 40 can define an exposed loop 120 extending from the top and bottom guide slots 82, 84. In some embodiments, the exposed loop 120 defines a relatively circular shape in side profile, and is longitudinally captured relative to the measuring tip/reference member 44 at the end guide slot 86 (e.g., the expansion member 40 cannot move distally beyond the slot 86). Regardless, the exposed loop 120 can be articulated to define a maximum distance D orthogonal to the longitudinal axis X of the sizing instrument 20. Stated otherwise, the expansion member 40 defines a maximum distance Dₜₒₚ from the top face 104 of the guide member 80 and a maximum distance D_{bottom} from the bottom face 106 of the guide member 80. In one embodiment, Dₜₒₚ is about equal to D_{bottom} such that the loop 120 is symmetrically arranged relative to the measuring tip 44. However, it should be understood that the slidably-captured interface between a distal portion of the loop 120 and the end guide slot 86 allows for non-symmetric or asymmetric arrangements of the loop 120 relative to the measuring tip 44 whereby where Dₜₒₚ is not equal to D_{bottom}. That is to say, in the presence of an external force (or overt resistance to transverse movement/deflection) upon one “side” of the expansion member 40, the distal portion of the loop 120 can slide relative to the end guide slot 86 such that the opposite “side” of the loop can experience further movement/deflection transversely relative to the measuring tip 44 beyond the orientation of FIG. 9. Thus, while the expansion member 40 is illustrated in FIG. 9 as having a symmetrical orientation relative to the measuring tip 44, the expansion member 40 is, in some embodiments, equally able to assuming, or being forced to, an asymmetrical orientation relative to the measuring tip 44.

With the arrangement of FIG. 9, the expansion member 40 effectively defines first and second legs or movable members or legs 40A and 40B relative to the measuring tip (or reference member) 44. With this in mind, in other embodiments, the first and second movable members 40A and 40B are separately provided and can independently move relative to the measuring tip 44. In other related embodiments, only one of the movable members or legs 40A or 40B is provided (and is movable relative to the measuring tip or reference member 44 in providing an indication of a dimension or spacing between the movable member 40A or 40B and the measuring tip 44).

In light of the above-described relationships, it should be understood that by actuating the piston 42 proximally and distally, the first and second end portions 46, 48 of the expansion member 40 are also moved proximally and distally. As the piston 42 is moved distally, the expansion member 40 (and in particular, the legs 40A and 40B) is expanded, or moved outwardly, transversely away from the top and bottom faces 104, 106 of the guide member 102, respectively. In some embodiments, the expansion member 40 is also moved away from the proximal end face 112 against the distal end face 114 of the guide member 102. In turn, moving the piston 42 proximally results in the expansion member 40 (and in particular the legs 40A and 40B) collapsing, or moving transversely inwardly, toward the top and bottom faces 104, 106 of the guide member 102, as well as toward the proximal end face 112 of guide member 102.

Along these lines, the piston 42 is movable proximally to a maximum proximal extent, or maximum proximal stroke permitted by the pin 56 as it travels in the top channel 94 and the bottom channel (not shown). In turn, the piston 42 is also movable distally to a maximum distal extent, or maximum distal stroke permitted by the pin 56 as it travels in the top channel 94 and the bottom channel (unnumbered). In one embodiment, at or before the piston 42 has traveled to the maximum proximal stroke, the expansion member 40 (and in particular the legs 40A and 40B) is pulled against the top and bottom faces 104, 106 of the guide member 102, as well as the proximal end face 112 of the guide member 102. In this manner, the expansion member 40 defines a minimized profile (FIG. 28) upon moving the piston 42 proximally a sufficient amount. For example, at the minimized profile, the maximum distance D (FIG. 9) is about equal to the thickness of the guide member 80, while the maximum distance Dₜₒₚ (FIG. 9) is about equal to the maximum distance D_{bottom} (FIG. 9), and in particular, both are about zero. In the absence of an external force (or resistance to transverse movement), the expansion member 40 (e.g., the legs 40A, 40B) is freely moveable to any spatial position or extent of deflection between the minimized profile and the maximum profile or distance D.

Returning to FIG. 2, the handle assembly 23 includes, in some embodiments, a guide assembly 24, a main handle 26, a spring 28, a drive tube 30, a translation shaft 32, and an adjustment handle 34. In general terms, the main handle 26 maintains, or is associated with, the remaining components 24, 28-34, and facilitates operation of the handle assembly 23 in actuating the expansion assembly 22.

With reference to FIG. 10, the guide assembly 24 includes a tip 130 and a guide tube 132. The tip 130 includes or forms a first stop 134 and a second stop 136 extending as distal protrusions from a base 138. The base 138 is substantially cylindrical in shape and defines an inner lumen 140 (referenced generally) and a plurality of snap fit projections 142.

The guide tube 132 between a proximal portion 146 to a distal portion 148, and has an inner lumen 150 configured to receive a portion of the main handle 26. The proximal portion 146 includes a base 152 and a grip collar 154. The base 152 includes a threaded surface 153 formed to mate with a corresponding surface portion of the main handle 26 (FIG. 2). The distal portion 148 forms an annular groove 156 about a circumference of the guide tube 132.

The guide tube 132 and the tip 130 are assembled by sliding the base 138 of the tip 130 over the distal portion 148 of the guide tube 132. In particular, the distal portion 140 of the guide tube 132 is coaxially received within the inner lumen 140 of the base 138. The plurality of snap-fit projections 142 are then mated or captured within the groove 156 of the distal portion 148 to secure the tip 130 to the guide tube 132. Upon final assembly, then, the first and second stops 134, 136 extend distally beyond the distal portion 148 of the guide tube 132.

With reference to FIG. 11, the main handle 26 includes a grip 162 and a tube 164. In general terms, the tube 164 is coaxially received within the grip 162. For example, as best shown in FIG. 12, the grip 162 defines an exterior or grip surface 168 and an inner lumen 170. The surface grip 168 is textured to facilitate grasping the grip 162. The inner lumen 170 has a proximal portion 174, an intermediate portion 176, and a distal portion 178. The grip 160 includes or forms first internal threads 180 at or along the proximal portion 172 and second internal threads 182 formed at or along the distal portion 178.

The proximal portion 174, and in particular the first threads 180, is adapted to mate with a portion of the adjustment handle 34 (FIG. 2). The intermediate portion 176 has a reduced diameter relative to the proximal and distal portions 174, 178, and is formed to coaxially receive and secure the tube 164 in the grip 162. The distal portion 178, and in particular the second threads 182, is adapted to mate with the base 152 of the guide assembly 24 (FIG. 2). As will be described in greater detail below, this interaction allows the guide assembly 24 to be moved distally and proximally relative to the main handle 26.

The tube 164 is substantially cylindrical, defines an outer circumference, and extends from a proximal end 186 to a distal end 188 (FIG. 11) at which an expansion assembly seat 190 (FIG. 11) is formed. The tube 164 also defines an inner lumen 192 extending from the proximal end 186 to the distal end 188. The distal end 188 of the tube 164 further forms internal threads (not shown) along the inner lumen 192. As will be described in greater detail below, the internal threads are formed to threadably engage a corresponding threaded surface of the first collar 72 (FIG. 5). In one embodiment, the tube 164 is sized to be coaxially receivable within the inner lumen 150 (FIG. 10) of the guide assembly 24 (FIG. 10). Additionally, in an assembled state or as formed, the proximal end 186 is coaxially received and secured within the intermediate portion 176 of the grip 162. In turn, the distal end 188, and in particular the expansion assembly seat 190, is configured to receive and abut against the proximal face 98 (FIG. 5) of the second collar 76 (FIG. 5).

Returning to FIG. 2, the spring 28 extends from a proximal end 194 to a distal end 196. In general terms, the spring 28 is selected to complement the flexibility of the expansion member 40. As will be described in greater detail below, a spring constant of the spring 28 is selected such that the spring 28 is deflected when the expansion member 40 (and in particular one or both of the legs 40A and/or 40B) meets a certain amount of resistance to expansion away from the guide member 80. Regardless, the proximal end 194 is adapted to abut against a portion of the drive tube 30, while the distal end 196 is adapted to abut against the spring seat 66 of the piston 42.

With reference to FIG. 13, the drive tube 30 can be an integrally formed component, and includes a shaft guide 200, a collar 204, and a body 206. The drive tube 30 also defines an inner lumen 208 (FIG. 14) extending through the shaft guide 200, the collar 204, and the body 206.

The shaft guide 200 is elongate and substantially cylindrical in shape, defining a proximal end 210, a first slot 212, a second slot 214, and an outer diameter. The proximal end 210 is formed to be received in a portion of the indicator assembly 36 (FIG. 2), as will be described in greater detail below. The first and second slots 212, 214 are disposed in an opposing manner and are formed longitudinally in the shaft guide 200. More particularly, the first and second slots 212, 214 extend distally from the proximal end 210 and are open to an exterior of the shaft guide 200, the inner lumen 208, and the proximal end 210. In general terms, the first and second slots 212, 214 are formed to receive a portion of the translation shaft 32 (FIG. 2). As will be understood with reference to the text that follows, the shaft guide 200, is adapted to be coaxially received by, and press fit in, a portion of the indicator assembly 36 in one embodiment.

The collar 204 is substantially cylindrical in shape and is disposed between the shaft guide 200 and the body 206. The collar 204 defines a proximal face 218 and a distal face 220 (FIG. 14). The shaft guide 200 extends proximally from the proximal face 218, while the body 206 extends distally from the distal face 220. As will be described in greater detail below, the proximal face 218 is adapted to abut against a portion of the actuation handle 34 (FIG. 2).

With reference to FIG. 14, the body 206 is substantially cylindrical in shape defining an outer diameter and extending to a distal end 222. The distal end 222 is configured to abut against the proximal end 194 of the spring 28 (FIG. 2), as will be described in greater detail below. The outer diameter of the body 206 is formed to allow the body 206 to be coaxially received in the inner lumen 192 of the main handle 26 (FIG. 11). In turn, the inner lumen 208 of the drive tube 30 is sized to receive a portion of translation shaft 32 (FIG. 2).

With reference to FIG. 15, the translation shaft 32 defines a proximal end 224 and a distal end 226. The translation shaft 32 includes a body 228, a tip 230, a first pin 232, and a second pin 234. The body 228 is solid and substantially cylindrical, defining an outer diameter. The body 228 includes a first transverse hole (not shown) proximate the proximal end 224 and a second transverse hole (not shown) distal the first transverse hole. The first and second holes are formed to receive the first and second pins 232, 234, respectively. The outer diameter is formed such that the body 228 is coaxially and slidably receivable in the inner lumen 208 of the drive tube 30 (FIG. 14), and in portions of the adjustment handle 34 (FIG. 2) and the indicator assembly 36 (FIG. 2), as will be described in greater detail below.

The tip 230 extends distally from the body 228 and is located at the distal end 226. The tip 230 forms threads 236 for threadably engaging a component of the expansion assembly 22 as described below.

The first and second pins 232, 234 are coaxially received, and secured, in the first and second holes (not shown) of the body 228. The first pin 232 is formed to be received in the first and second slots 212, 214 of the drive tube 30. The second pin 234 is formed to be received in a portion of the indicator assembly 36 (FIG. 2), as will be described in greater detail below.

With reference to FIG. 16, the adjustment handle 34 can be formed as a single piece and defines a proximal end 242 and a distal end 244. The adjustment handle 34 forms a grip 246, a distal portion 248, and an inner lumen 250. The distal end 244 is configured to abut against the collar 204 (FIG. 13), as will be described in greater detail below. The grip 246 is textured to facilitate grasping the grip 246. The distal portion 248 forms threads 250 to threadably mate with a corresponding surface of the main handle 26 (FIG. 11).

As best shown in FIG. 17, the inner lumen 250 is stepped. In particular, the inner lumen 250 has a greater diameter at the grip 246 than at the threaded portion 248. In one embodiment, the inner lumen 250 is sized along the grip 246 to coaxially receive a portion of the indicator assembly 36 (FIG. 2), as will be described in greater detail below. In turn, the inner lumen 250 at the distal portion 248 is sized to coaxially receive a portion of the drive tube 30 (FIG. 2).

Returning to FIG. 2, in some embodiments, the indicator assembly 36 includes an insert piece 260, a scale cap 262, and a scale pointer 264. As best shown in FIG. 18, the insert piece 260 defines a proximal end 268 and a distal end 270, and forms a base 272, a distal projection 274, and an inner lumen 276 extending through the base 272 and the distal projection 274.

The base 272 forms the proximal end 268, is substantially cylindrical, and defines an outer circumference. The base 272 includes a grip surface 278, a groove 280, and a mating collar 282. The grip surface 278 is textured to facilitate grasping the insert piece 260. The groove 280 is formed about an outer circumference of the base 272 adjacent the proximal end 268 and is sized to capture a corresponding surface of the scale cap 262. The mating collar 282 is located proximal the groove 280. The mating collar 282 defines an outer diameter, the outer diameter of the mating collar 282 greater than that of the groove 280. The mating collar 282 is also formed to be received by the scale cap 262.

The distal projection 274 extends from the base 272 to the distal end 270, defines an outer diameter, and forms a first slot 288 and a second slot 290 (partially obscured in FIG. 18). The outer diameter of the distal projection 274 is formed to allow the distal projection 274 to be coaxially received in the inner lumen 250 of the adjustment handle 34.

The first slot 288 extends longitudinally from the distal end 270 to a terminal end 294. The second slot 290 also extends longitudinally from the distal end 270 to the terminal end 294. In relational terms, the first and second slots 288, 290 are opposingly formed in the distal projection 274. Furthermore, the first and second slots 288, 290 are open to an exterior of the distal projection 274, the inner lumen 276, and the distal end 270. As will be understood in greater detail below, the first and second slots 288, 290 are formed to receive the second pin 232 of the translation shaft 32 (FIG. 2).

As best shown in FIG. 19, the inner lumen 276 is formed in a stepped fashion such that the inner lumen 276 has a first portion 298, a second portion 300, and a third portion 302. The first portion 298 is open at the distal end 270 and is sized to receive the drive tube 30 (FIG. 2). The second portion 300 is sized to receive the translation shaft 32 (FIG. 2). The third portion 302 is sized to receive the scale pointer 264 (FIG. 2).

With reference to FIGS. 20 and 21, the scale cap 262 is cylindrical, defines a distal face 308 (FIG. 21), and forms an insert seat 310, a pointer seat 312, and a dial seat 314. In general terms, the insert seat 310 is configured to receive the insert piece 260 (FIG. 2). In turn, the pointer seat 312 and the dial seat 314 are configured to receive a portion of the scale pointer 264 (FIG. 2), as will be described in greater detail below. In some embodiments, the distal face 308 and/or the pointer seat 312 includes indicia (not shown) conveying dimensional information as described below.

With additional reference to FIG. 22, the insert seat 310 has a first bore portion 320 and a second bore portion 322. The first bore portion 320 is complementary in shape to the groove 280 of the insert piece 260 (FIG. 18). The second bore portion 322 is complementary in shape to the mating collar 282 of the insert piece 260 (FIG. 18). The pointer seat 312 has a first slot segment 324, a second slot segment 326, and a third slot segment 328. As will be described in greater detail below, the segments 324, 326, and 328 are formed to coaxially, and rotatably, receive various portions of the scale pointer 264 (FIG. 2). Regardless, and as best shown in FIGS. 20 and 21, the bore portions 320, 322, and the slots segments 324-328 are transversely open along a side of the scale cap 262.

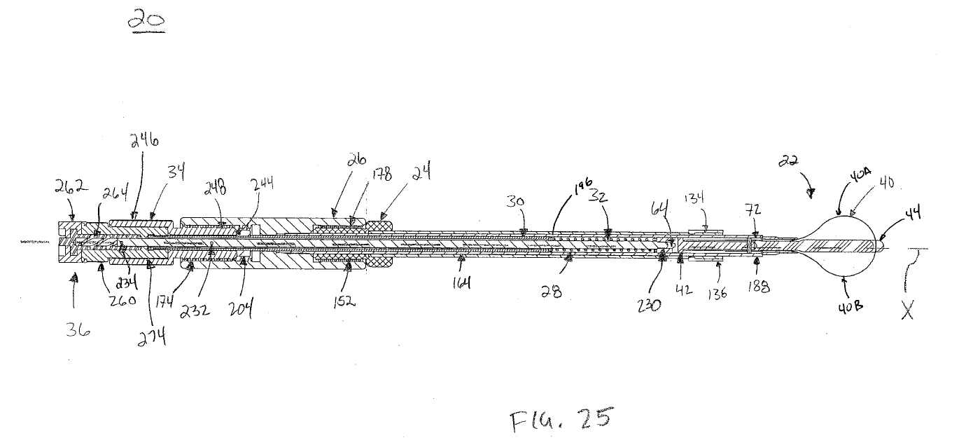

With reference to FIGS. 23 and 24, the scale pointer 264 can be an integral body and includes a shaft 330, a collar 332, a neck 334, and a pointer 336. The shaft 330 has a double helix configuration, defining a first pin path 342 and a second pin path 344. In particular, the first and second pin paths 342, 344 are sized to slidably receive the first pin 234 (FIG. 15) of the translation shaft 32 (FIG. 15). In one embodiment, the collar 332 is cylindrical and defines an outer diameter greater than the shaft 330. The neck 334 is also cylindrical and defines an outer diameter less than that of the collar 332. Finally, the pointer 336 can have a triangular shape and extends orthogonally from the neck 334.

Assembly of the instrument 20 can be described reference to FIG. 25. In general terms, the expansion assembly 22, the handle assembly 23, and the indicator assembly 36 are all coaxially disposed relative to one another along the longitudinal axis X.

With this in mind, the first collar 72 of the expansion assembly 22 is threadably secured to the distal end 188 of the tube 164 of the main handle 24. In terms of removability, the expansion assembly 22 can be unscrewed from the distal end 188 and used in a single-use application. The base 152 of the guide assembly 24 is threadably secured to the distal portion 178 of the main handle 26. With this configuration, the base 152 can be rotated relative to the distal portion 178 to distally or proximally adjust, or vary a position of, the first and second stops 134, 136 relative to the expansion assembly 22.

The spring 28 is coaxially received within the tube 164 of the main handle 26. Generally, the distal end 196 (FIG. 2) of the spring 28 is abutted against the piston 42 of the expansion assembly 22. More particularly, the distal end 196 is received in the spring seat 66 (FIG. 4) of the piston 42. The drive tube 30 is coaxially received within the grip 162 and the tube 164 of the main handle 26. In particular, the distal end 222 (FIG. 14) of the drive tube 30 is abutted against the proximal end 194 of the spring 28. The collar 204 of the drive tube 30 is coaxially received in the proximal portion 174 of the grip 162 of the main handle 26. With this in mind, distal movement of the drive tube 30 is translated to the spring 28, which, in turn, exerts a distal force on the piston 42 of the expansion assembly 22.

The translation shaft 32 is the coaxially received within the drive tube 30, and arranged such that the tip 230 is coaxially received through the spring 28 and projects into the cavity 64 of the piston 42. In this regard, the tip 230 is threadably secured to the cavity 64. In one embodiment, after the tip 230 has been threadably engaged with the cavity 64, the first pin 232 of the translation shaft 32 is received in the first and second slots 212, 214 (FIG. 13) of the drive tube 30. In this manner, proximal and distal movement of the piston 42 or the translation shaft 32 results in distal or proximal movement of the other. With the above in mind, the expansion assembly 22, the main handle 26, the drive tube 30, and the translation shaft 32 are no longer substantially rotatable relative to one another without disassembling the parts, although the translation shaft 32 is free to move distally and proximally relative to the spring 28 and the drive tube 30.

The distal portion 248 of the adjustment handle 34 is threadably mounted to the proximal portion 174 of the main handle 26. With this arrangement, rotation of the adjustment handle 34 results in distal or proximal movement of the adjustment handle 34 relative to the main handle 26.

The insert piece 260 of the indicator assembly 36, and in particular the distal projection 274, is coaxially and rotatably received in the grip portion 246 of the adjustment handle 34. Additionally, the shaft guide 200 (FIG. 13) of the drive tube 30 is press fit into the first portion 298 (FIG. 19) of the insert 260, with the first and second slots 288, 290 (FIG. 18) of the insert piece 260 aligned to the first and second slots 212, 214 (FIG. 13) of the drive tube 30. From this, it should be understood that the first pin 232 of the translation shaft 32 is received, and slidable in portions of both the first slots 212, 288 and the second slots 214, 290. In particular, the first slots 212, 288, combine to define a first combined slot (not shown) extending from a proximal end to a distal end while the second slots 214, 290 combine to define a second combined slot extending from a proximal end to a distal end. The first combined slot is disposed in an opposing manner to the second combined slot.

In terms of relative movement, the insert piece 260 moves proximally and distally with the drive tube 30, and is rotationally fixed relative to the drive tube 30. However, the insert piece 260 and the drive tube 30 are free to rotate relative to the adjustment handle 34. Furthermore, the translation shaft 32 is free to move proximally and distally within the first and second combined slots (not shown).

The scale pointer 264, and in particular a portion of the shaft 330 (FIG. 24) is coaxially and rotatably received in the third portion 302 (FIG. 19) of the insert 260. In turn, the second pin 234 of the translation shaft 32 is received in the first and second pin paths 340, 342 (FIG. 24) of the shaft 330, such that proximal or distal movement of the translation shaft 32 relative to the shaft 330 induces rotation of the scale pointer 264 that is proportionate to an amount of the proximal and/or distal movement of the translation shaft 32.

The scale cap 262 is used, in part, to assist in preventing the scale pointer 264 from moving distally or proximally relative to the insert piece 260, while still allowing the scale pointer 264 to rotate relative to the insert 260 and the scale cap 226. In particular, the groove 280 (FIG. 19) and the mating collar 282 (FIG. 19) of the insert piece 260 are coaxially received in, and secured to, the insert seat 310 (FIG. 22) of the scale cap 262. For example, the scale cap 262 is snap fit over the groove 280 and the mating collar 282. Additionally, a portion of the shaft 330 (FIG. 23) is coaxially and rotatably received in the first portion 324 of the pointer seat 312 (FIG. 22). The collar 332 (FIG. 23) of the scale pointer 264 is coaxially and rotatably received in the second portion 326 (FIG. 22) of the pointer seat 312. The neck 334 (FIG. 23) of the scale pointer 264 is coaxially and rotatably received in the third portion 328 (FIG. 22) of the pointer seat 312. Additionally, the pointer 336 (FIG. 23) is coaxially and rotatably received in the dial seat 314 (FIG. 22).

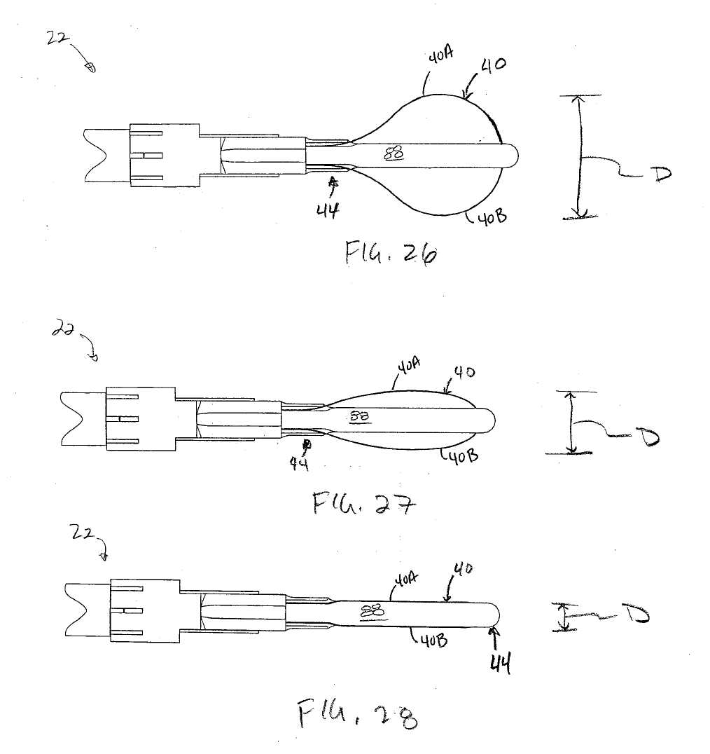

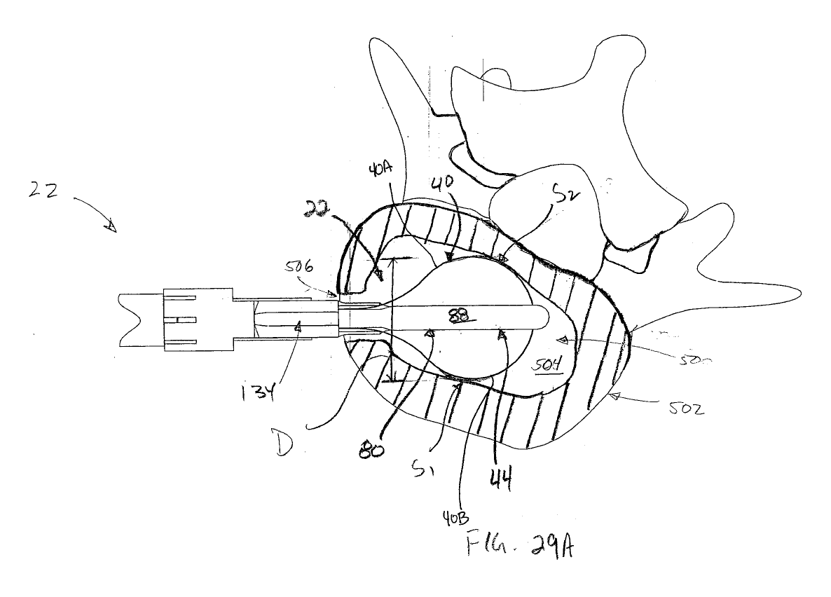

In view of the above, operation, or actuation, of the sizing instrument 20 includes rotating the adjustment handle 34 relative to the main handle 26. In one embodiment, the adjustment handle 34 is rotated a first direction to move the adjustment handle 34 distally relative to the main handle 26. With sufficient distal movement, the distal end 244 of the adjustment handle 34 abuts against the collar 204 of the drive tube 30, moving the drive tube 30 distally. As a result, distal force is applied to the spring 28, which is translated to the piston 42 of the expansion assembly 22. If there is sufficient force to overcome any resistance to expansion encountered by the expansion assembly 22, the piston 42 will also move distally, expanding the expansion member 40 to various amounts of expansion (FIGS. 26-28), for example from a first unexpanded state (FIG. 28) to a first expanded state (FIG. 27), or from the first expanded state (FIG. 27) to a second expanded state (FIG. 26). In some embodiments (e.g., where the expansion member 40 is a strip), transitioning or movement thereof relative to the measuring tip 44 is characterized by a proximal portion of the expansion member 40 being forced distally (or proximally) and an intermediate portion of the expansion member 40 deflecting transversely outwardly (or inwardly) relative to the measuring tip 44. However, if the expansion assembly 22 encounters sufficient resistance to expansion and/or further expansion, the spring 28 will simply be compressed without movement of the piston 42 as the adjustment handle is turned in the first direction. If the spring 28 is compressed sufficiently to overcome any such resistance, distal movement of the piston 42 can begin again in some embodiments.

As the piston 42 is moved distally, the translation shaft 32 is also moved distally. Distal movement of the translation shaft 32 is translated into rotation of the scale pointer 264 as the second pin 234 of the translation shaft 32 is moved distally within the shaft 330 (FIG. 24) of the scale pointer 264. In this manner, an amount of rotation of the scale pointer 264 corresponds to an amount of expansion of the expandable member 40.

In one embodiment, rotating the adjustment handle 34 in an opposite, second direction results in proximal movement of the adjustment handle 34 relative to the main handle 26. The adjustment handle 34 presses proximally against the insert piece 260, which as previously described is secured to the drive tube 30. Thus, as the adjustment handle 34 moves proximally, so does the drive tube 30. The first pin 232 of the translation shaft 32 travels within the first and second combined slots (not shown) to a point where the first pin 232 contacts the distal ends (not shown) of the first and second combined slots. Upon further proximal movement of the drive tube 30 and the insert piece 260, the translation shaft 32 is also moved proximally. Proximal movement of the translation shaft 32 initiates collapsing of the expansion assembly 22. Proximal movement of the translation shaft 32 is also translated into rotation of the scale pointer 264. Thus, the scale pointer 264 also reflects a relative amount of expansion of, or collapsing of, the expansion member 40 in some embodiments. Ultimately, upon sufficient proximal movement of the translation shaft 32, the expansion assembly 22 is transitioned to a fully collapsed state (FIG. 28) with the expansion member 40 (and in particular the legs 40A and 40B) collapsed against the measuring tip 44 (FIG. 28) to define a minimized profile.

In one embodiment, the distal face 308 (FIG. 21) of the scale cap 262 includes indicia 400 (FIG. 29B) to communicate the amount of expansion of the expansion member 40 to a user at a location spaced from, or outside of, the bodily joint being evaluated. For example, the pointer 336 (FIG. 24) rotates to point at a number (or other indicia 400) corresponding to, or understood by a user as being indicative of, a dimensional measurement as the translation shaft 32 is moved proximally and distally. Depending upon an orientation of the expansion member 40 relative to the bodily joint being evaluated, the value indicated by the pointer 336/indicia 400 can be indicative of a number of different dimensions of the bodily joint. For example, the indicia correspond to a width measurement, a height measurement, an area measurement, etc.

The instrument 20 can be employed to perform measuring or sizing operations for a wide variety of applications, such as part of surgical procedures. The instrument 20 is of particular usefulness in measuring or sizing a confined area, in particular a bodily joint generally having opposed, upper and lower surfaces. With these applications, the instrument 20, and in particular the expansion assembly 22, can be initially actuated to a low profile for insertion into the bodily joint. With this low profile arrangement, then, only a minor or small opening into the bodily joint is required. However, the expansion assembly 22 readily expands within the joint, with the handle assembly 23/indicator assembly 36 facilitating manipulation of the expansion assembly 22 as well as indicating to the clinician a dimension being “measured” by the expanded expansion member 40.

By way of one example, FIG. 29A illustrates a portion of the instrument 20 inserted into an intervertebral disc space 500 (it being recalled that the instrument 20 can be used to measure or size a number of other bodily joints, such as the shoulder, knee, etc.). By way of background, the intervertebral disc space 500 is bounded by an annulus 502, a top vertebra (not shown), and a bottom vertebra 504. Nucleus material (not shown) may or may not be present in the intervertebral disc space 500. A hole 506 is show in the annulus 502, with expansion assembly 22 having been inserted into the intervertebral disc space 500 through the hole 506.

As a point of reference, prior to insertion within the disc space 500, the expansion member 40 is collapsed to a fully collapsed state (FIG. 28), or minimized profile, such that it lies flat against the measuring tip 44. As described above, such collapsing or moving of the expansion member 40 (and in particular the movable legs or members 40A, 40B) relative to the measuring tip or reference member 44 is accomplished by rotating the adjustment handle 34 (FIG. 25) in the second direction.

The hole 506 is formed in the annulus 502, for example via methods and instruments (not shown) known to those of skill in the art. The disc nucleus (not shown), or a portion thereof is removed from the intervertebral disc space 500. The measuring tip 44 is advanced into the hole 506 until the guide assembly 24 (FIG. 16), and in particular the first and/or second stops 134, 136 (FIG. 10) come into contact with the annulus 502, the top vertebra, and/or the bottom vertebra 504. In particular, the guide assembly 24 is adjusted proximally or distally relative to the expansion assembly 22 to pre-select a distance that the measuring tip 44 is advanced into the intervertebral disc space 500.

Once the measuring tip 44 has been advanced into the intervertebral disc space 500 as desired, for example the pre-selected distance, the adjustment handle 34 (FIG. 25) is rotated, or actuated, in the first direction to move or expand the expansion member 40 (in particular the legs 40A, 40B) relative to (away from) the measuring tip 44, and in particular the guide member 80 of the measuring tip 44. In turn, the scale pointer 264 of the indicator assembly 36 rotates to indicate a dimension corresponding to an amount of expansion of the expansion member 40 as schematically illustrated in FIG. 29B. Eventually, and returning to FIG. 29A, the expansion member legs 40A, 40B contact the annulus 502 along one or more contact surfaces S1, S2.

In some embodiments, expansion of the expansion member 40 stops and the spring 28 (FIG. 24) begins compressing due to the resistance to further expansion encountered by the expansion member 40 (e.g., when one or both of the legs 40A and/or 40B moves into contact with a structure associated with the bodily joint being measured or sized). For example, the expansion member leg(s) 40A and/or 40B begin to conform and deflect to the shape of the annulus 502 along the corresponding contact surfaces S₁, S₂ until sufficient resistance occurs to compress the spring 28. Along these lines, the spring 28 begins to compress, and the expansion member 40 begins to deflect until the expansion member 40 encounters sufficient resistance to further expansion such that only compression of the spring 28 occurs. Regardless, once expansion of the expansion member legs 40A, 40B is arrested, the scale pointer 264 (FIG. 29B) ceases to turn, and a dimensional measurement of the joint region in question is communicated to a user (not shown).

In some embodiments, the dimensional measurement corresponds to the maximum distance D defined by the exposed loop 120. In other embodiments, the dimensional measurement is an area measurement. For example, an area substantially “enclosed” by the exposed loop 120 is the dimensional measurement taken. The area measurement is particularly useful where the expansion member 40 is flexible enough to substantially conform to the annulus 506 (or other bodily joint structure) during expansion, such that an estimated transverse area of the intervertebral disc space 500 is measured and communicated to the user. It should also be understood that the instrument 20 can be rotated, for example about 90 degrees from the position shown in FIG. 29A, to measure a distance between the top vertebra and the bottom vertebra 504. For example, in one embodiment, a height of the intervertebral disc space 500 is the dimensional measurement taken.

Regardless, following taking of the dimensional measurement, the adjustment handle 34 (FIG. 25) is rotated, or actuated in the second direction to collapse the expansion assembly to the fully collapsed state (FIG. 28). Finally, the expansion assembly 22 is removed from the intervertebral disc space 500, for example by pulling proximally on the grip 162 (FIG. 25) of the main handle 26 (FIG. 25). In one embodiment, the expansion assembly 22 is unscrewed from the instrument 20, and a second expansion assembly (not shown) is attached to the instrument 20. In this manner, the expansion assembly 22 can be used in a single-use application.

Although specific embodiments have been illustrated and described herein, it will be appreciated by those of ordinary skill in the art that a variety of alternate and/or equivalent implementations may be substituted for the specific embodiments shown and described without departing from the scope of the present invention. This application is intended to cover any adaptations or variations of the specific embodiments discussed herein. Where reference is made to directional terminology, e.g., “top,” bottom,” “front,” “back,” “left,” “right,” it should be understood such reference is to the orientation of the figure(s) being described. Therefore, it is intended that this invention be limited only by the claims and the equivalents thereof. Thus, while the sizing instrument has been described as having certain components/assemblies, a wide variety of other constructions can be employed. In more general terms, aspects of the present invention reside in a sizing instrument, a distal portion of which includes a reference (or fixed) member and at least one moveable member that can be actuated to move transversely relative to the reference member. The amount or level of movement is directly translated to a proximal end of the instrument, and is conveyed to a user.

What is claimed is:

1. A surgical sizing instrument for internally measuring a bodily joint, the instrument comprising:
an expansion assembly including a reference member and a movable member, the expansion assembly configured such that the movable member is transversely transitionable relative to the reference member from a first position to a second position;
a handle assembly maintaining the expansion assembly at a distal portion thereof and adapted to actuate the movable member between the first position and the second position; and
an indicator assembly associated with a proximal portion of the handle assembly and including an indicator adapted to communicate a dimensional measurement associated with the second position.

2. The instrument of claim 1, wherein the instrument is configured to provide sizing information of an intervertebral disc space.

3. The instrument of claim 1, wherein the handle assembly is configured to translate a movement of the moveable member relative to the reference member to the indicator assembly.

4. The instrument of claim 1, wherein the movable member is a transversely flexible strip.

5. The instrument of claim 4, wherein the strip is substantially rectangular in lateral cross-section.

6. The instrument of claim 4, wherein the strip is substantially circular in lateral cross-section.

7. The instrument of claim 1, wherein the expansion assembly is configured such that the moveable member is freely transitionable relative to the reference member to any transverse position between the first and second positions.

8. The instrument of claim 1, wherein the expansion assembly is configured such that a distal portion of the moveable member is longitudinally captured relative to the reference member, whereas a proximal portion of the moveable member is longitudinally moveable relative to reference member.

9. The instrument of claim 8, wherein the expansion assembly is configured such that as the proximal portion of the moveable member is moved distally relative to the reference member, an intermediate portion of the moveable member deflects transversely outwardly relative to the reference member.

10. The instrument of claim 1, wherein the moveable member forms a loop relative to the reference member to define first and second legs.

11. The instrument of claim 10, wherein the expansion assembly is configured such that the legs are asymmetrically moveable relative to the reference member.

12. The instrument of claim 10, wherein a distal portion of the moveable member is a loop end and is slidably connected to the reference member.

13. The instrument of claim 10, wherein the expansion assembly is configured such that in the first position, the legs conform to a shape of the reference member.

14. The instrument of claim 10, wherein the legs combine to define a discontinuous loop.

15. The instrument of claim 1, wherein the expansion assembly further includes a piston maintaining the proximal portion of the moveable member such that longitudinal movement of the piston dictates a position of the moveable member relative to the reference member.

16. The instrument of claim 15, wherein the handle assembly includes an adjustment handle and is adapted to translate a rotational movement of the adjustment handle into an axial movement of the piston.

17. The instrument of claim 16, wherein the handle assembly further includes a drive rod connected to the adjustment handle and a spring disposed between the drive rod and the piston, the spring generating a bias force such that in the presence of a first level of resistance to movement of the movable member relative to the reference member, the spring permits translation of a distal movement of the drive rod to the piston, and in the presence of a second level of resistance greater than the first level, the spring prevents translation of a distal movement of the drive rod to the piston.

18. The instrument of claim 1, wherein the indicator assembly includes scaled indicia with which the indicator is movably associated.

19. A method of measuring an interior of bodily joint, the method comprising:
providing a sizing instrument comprising:
an expansion assembly including a reference member and a movable member, the expansion assembly configured such that the movable member is transversely transitionable relative to the reference member from a first position to a second position,
a handle assembly maintaining the expansion assembly at a distal portion thereof and adapted to actuate the movable member between the first position and the second position,
an indicator assembly associated with a proximal portion of the handle assembly and including an indicator adapted to communicate a dimensional measurement associated with the second position,
collapsing the movable member transversely toward the reference member,
disposing the reference member and the moveable member in the bodily joint, the bodily joint having a dimension;
expanding the movable member relative to the reference member and against a boundary of the bodily joint to measure the dimension; and
communicating the dimension via the indicator assembly.

20. The method of claim 19, wherein the bodily joint is an intervertebral disc space.

21. The method of claim 20, further comprising:
measuring a width dimension of the intervertebral disc space with the instrument; and
measuring a height dimension of the intervertebral disc space with the instrument;
wherein the width and height dimensions are measured without removing the movable member from the disc space.

22. The method of claim 19, wherein expanding the movable member relative to the reference member includes manually rotating an adjustment handle of the handle assembly.

23. The method of claim 19, wherein expanding the movable member includes the movable member conforming to a shape of the boundary against which the movable member is disposed.

24. The method of claim 19, wherein the expansion assembly includes a strip forming a looped connection with the reference member to define the movable member as including first and second legs, and further wherein expanding the moveable member includes a distance between the first and second legs being indicative of the dimension.

25. The method of claim 19, further comprising:
removing the expansion assembly from the handle assembly; and
securing a second, different expansion assembly to the handle assembly.
